(19) United States
(12) Patent Application Publication
(10) Pub. No.: US 2004/0156801 A1
(43) Pub. Date: Aug. 12, 2004

(54) Nail softener for use in deformed-nail corrective treatment or nail-structure modification, enclosure for external chemicals and method of correcting deformed nail

(76) Inventors: Kikuzo Okada, Tokyo (JP); Yoko Okada, Tokyo (JP)

Correspondence Address: Edward G. Greive, Renner, Kenner, Greive, Bobak, Tayor & Weber, Fourth Floor, First National Tower, Akron, OH, 44308-1456, US

(21) Appl. No.: 10/770,259

(22) Filed: Feb. 2, 2004

(30) Foreign Application Priority Data
Feb. 3, 2003 (JP) 2003-025831
Feb. 3, 2003 (JP) 2003-025832

(51) Int. Cl. A61K 7/04

(52) U.S. Cl. 424/61

(57) ABSTRACT

Disclosed is an enclosure for an external chemical, which comprises an enclosure body receiving the external chemical therein and having an opening for exposing the external chemical when used, a sealing film for allowing the external chemical to be sealingly enclosed in the external chemical, an adhesive layer provided at the peripheral portion of the opening of the enclosure body, and a releasable film releasably attached to the enclosure body by use of the adhesive layer. In this enclosure, after the releasable film is removed, the adhesive layer serves as an adhesive portion for allowing the enclosure body to be attached to a diseased area. The enclosure of the present invention can reliably maintain the contact between the external chemical and the disease area in a simple structure.

### FIELD OF THE INVENTION

The present invention relates to a nail softener for use in deformed-nail corrective treatment or nail-structure modification, and a method of correcting a deformed nail. The present invention also relates to an enclosure for external chemicals, such as a nail softener for use in deformed-nail corrective treatment or nail-structure modification, or various kinds of drug including an antifungal drug or antimycosis drug, or a mixture thereof.

### BACKGROUND OF THE INVENTION

A deformed nail herein means a curved nail or an ingrown nail. For example, the deformed nail means the state after one or both of the lateral edges of a toenail grow while inwardly curving and cutting into the skin between the front edge of the toenail and the tip of the toe. In order to curing an ingrown nail, there theretofore have been proposed various methods depending on the level of the symptoms of ingrown nail. In case of nails having serious deformity, a method based on a surgical operation has been employed. There have also been proposed several ingrown-nail correction methods involving no surgical operation. Among them, one method comprises attaching a plate-shaped member made, for example, shape-memory alloy or resin, onto the curved surface of an ingrown nail using an adhesive, and increasing an ambient temperature up to a given temperature to correct the ingrown nail by means of a restoring force of the plate-shaped member (see Japanese Patent Publication No. 2648735). Another method comprises attaching a superelastic alloy wire between two holes formed in the front end of a deformed nail to correct the deformed nail by means of a gentle/gradual restoring force of the wire with a long period of several months.

Japanese Patent Laid-Open Publication No. 2001-276104 also discloses a correction tool for deformed nails and a correction method using the corrective tool as described below.

The deformed-nail correction tool comprises a first correction member, a second correction member and a correction-actuating portion. The first correction member includes a first engagement portion adapted to be engaged with one of the lateral edges of a nail, a first contact portion adapted to be brought into a portion of the surface of the nail, and a first connecting hook connected to the first contact portion. The second correction member includes a second engagement portion adapted to be engaged with the other lateral edge of the nail, a second contact portion adapted to be brought into a portion of the surface of the nail, and a second connecting hook connected to the second contact portion. The correction-actuating portion is adapted to be engaged with the first and second connecting hooks to pull both the lateral edges of the nail upwardly while holding the first and second engagement portions such that they are pulled toward the laterally intermediate portion of the nail.

The conventional method based on a surgical operation is required to incise in a wide range of skin and remove not only a deformedly curved portion of a nail but also a part of the matrix or root of the nail. Thus, a patient has to bear with intraoperative and postoperative pains and live with a permanently narrowed nail. Further, after the operation, the patient is required to wait for a suture removal for about two weeks and have injections of or orally take antibiotics.

In addition, even if an ingrown nail is temporarily corrected through a surgical operation of partially removing the nail, there is a high possibility that an ingrown nail will be developed in a regrown nail after the operation.

Among the conventional methods involving no surgical operation, in the method using a restoring force of the plate-shaped member made of shape-memory alloy, the restoring force cannot be effectively obtained in a region out of the elastic region of the shape-memory member. Further, this method uses an adhesive for attaching the plate-shaped member onto the surface of a nail. Thus, if the adhesive force of the adhesive is insufficient, the plate-shaped member is likely to peel off from the surface of the nail. Conversely, an excessively high adhesive force is likely to hinder the restoring force of the plate-shaped member.

The method using the correction tool as disclosed in the above Japanese Patent Laid-Open Publication No. 2001-276104 is required to take a long treatment period of several months as with the above method using the plate-shaped member. Thus, this corrective treatment obliges a patient to endure a hard time to live with the attached correction tool.

In the method using the superelastic alloy wire to be attached between two holes formed in the front end of an ingrown nail to correct the ingrown nail by means of a gentle/gradual restoring force of the wire, the restoring force is insufficient to a deformed nail having a high hardness. Further, the wire is apt to be readily detached during a long treatment period, and thus it is required to reattach the wire repeatedly.

It is therefore an object of the present invention to provide a nail softener for use in deformed-nail corrective treatment or nail-structure modification, capable of correcting a deformed nail, such as an ingrown nail, in a significantly short period of time (e.g. 1 hour to 1 day) through a simple process without any surgical operation. This nail softener is used not only for correcting the deformation of a nail but also for modifying the inside structure of a nail. In connection with nail treatments, a nail fungal infection or a nail mycosis (onychomycosis) recently occurs at a high level, and its treatment is enmeshed in difficulties. Even if a curative drug, such as an antifungal drug or antimycosis drug, is simply applied onto a local portion of a nail, any effect cannot be obtained to this symptom due to no infiltration of the curative drug into the hard structure of the nail. Practically, an expensive oral drug entailing adverse side effects such as hepatopathy has to be used for a long period of time. In view of avoiding such adverse side effects, there is a strong need for developing an effective external drug for the nail fungal infection or the nail mycosis, capable of locally acting on a nail.

From this point of view, the nail softener of the present invention can be effectively used with a curative drug such as antifungal drug or antimycosis drug. That is, when the nail softener and the curative drug are applied onto a local portion of a nail individually or in the form of a mixture thereof, the nail softener causes the change in the hard structure of the nail to allow the curative drug to infiltrate into the nail and effectively act on the nail fungal infection or nail mycosis.

In view of strengthening the effectiveness of an external chemical, such as the nail softener, and the antifungal drug or antimycosis drug, it is desirable to maintain the contact between the external chemical and a diseased area for a given period of time. However, only an existing method is to apply the external chemical onto a diseased area and cover the area using gauze, and thus it is required to develop a new instrument capable of reliably maintaining the contact between the external chemical and the diseased area. In particular, this need has been elicited in response to the achievement of the nail softener of the present invention.

It is therefore another object of the present invention to provide an enclosure for external chemicals, capable of maintaining the contact between an external chemical and a diseased area.

### SUMMARY OF THE INVENTION

Through various researches based on the conception that technologies of permanent wave could be applied to a corrective treatment for deformed nails because a nail has components similar to those of a hair, the inventors who are orthopedic surgeons had a knowledge that a nail can be softened using a reducing agent as a primary component of the first agent for permanent wave. That is, as with hairs, the disulfide bond of cystine contained in keratin protein of a nail is deoxidized and split as a mercapto group so as to soften a diseased nail (deformed nail) to correct the nail.

Specifically, in order to achieve the above object, according to a first aspect of the present invention, there is provided a nail softener for correcting a deformed nail or modifying the structure of a nail modification. The nail softener contains at least one reducing agent selected from the group consisting of cysteine, thioglycolic acid and thioglycolate.

In the nail softener set forth in the first aspect of the present invention, the reducing agent may be thioglycolate. In this case, the reducing agent may be ammonium thioglycolate.

In the above nail softener, the reducing agent may be subjected to a deodorizing treatment.

According to a second aspect of the present invention, there is provided an enclosure for an external chemical, comprising an enclosure body receiving the external chemical therein and having an opening for exposing the external chemical when used, a sealing film for allowing the external chemical to be sealingly enclosed in the external chemical, an adhesive layer provided at the peripheral portion of the opening of the enclosure body, and a releasable film releasably attached to the enclosure body by use of the adhesive layer. In this enclosure, after the releasable film is removed, the adhesive layer serves as an adhesive portion for allowing the enclosure body to be attached to a diseased area.

In the enclosure set forth in the second aspect of the present invention, the sealing film may be attached to the enclosure body by use of the adhesive layer so that the releasable film is served by the sealing film.

The sealing film may be adapted to be broken or pulled out of the enclosure body so as to allow the external chemical enclosed in the enclosure body to be exposed to the outside thereof.

The opening may have a size equal to or greater than that of the diseased area.

The enclosure body may include a ring-shaped pad, and an external-chemical enclosing pocket located on the top surface of the ring-shaped pad.

The external chemical may contain at least one reducing agent selected from the group consisting of cysteine, thioglycolic acid and thioglycolate. In this case, the reducing agent may be thioglycolate. Further, the reducing agent may be ammonium thioglycolate.

In the above enclosure, the reducing agent may be subjected to a deodorizing treatment.

In the enclosure set forth in the second aspect of the present invention, the external chemical may be a mixture of at least one reducing agent selected from the group consisting of cysteine, thioglycolic acid and thioglycolate, and a curative drug for the diseased area.

Alternatively, the external chemical may be a curative drug for the diseased area.

According to a third aspect of the present invention, there is provided a method of correcting a deformed nail, comprising the steps of (i) applying a nail softener containing at least one reducing agent selected from the group consisting of cysteine, thioglycolic acid and thioglycolate, onto a diseased area of a toenail, (ii) leaving the diseased area untouched for a given period of time to soften the nail, (iii) during or after the leaving step, applying a given force onto the nail to reposition the nail to its normal state, and (vi) immobilizing the nail in the repositioned state for a given period of time to hold the repositioned state.

As compared to conventional the techniques of applying a cream or ointment onto the surface of a nail, and covering the surface of the nail using gauze, the hermitically sealed enclosure allows the external chemical enclosed therein as with an occlusive dressing therapy to be effectively absorbed into and brought into contact with a diseased area, particularly the surfaces of the nail.

The external chemical enclosed in the enclosure can act only to the (front and rear) surfaces of the nail but not to any surrounding skin, to prevent the surface roughness of the skin from occurring.

Further, even if the enclosed external chemical has an odor, such an odor will be never diffused to surroundings.

If the enclosure is attached with an adhesive onto a toe, the external chemical will neither contact nor stain garments or bedclothes during body movement or in a dorsal position at night. Further, the enclosure may be made of a material having high sealing performance to allow a patient to take a bath while attaching the enclosure.

Other features and advantages of the present invention will be apparent from the accompanying drawings and from the detailed description.

### BRIEF DESCRIPTION OF THE DRAWINGS

FIG. 1 is an explanatory diagram of an ingrown-nail correcting method using a nail softener according to one embodiment of the present invention, particularly of a process of forming holes in a nail.

FIG. 2 is another explanatory diagram of an ingrown-nail correcting method using a nail softener according to one embodiment of the present invention, particularly of a process of forming holes in a nail.

FIG. 3 is another explanatory diagram of an ingrown-nail correcting method using a nail softener according to one embodiment of the present invention, particularly of a process of fixing a nail using resin.

FIG. 4 is explanatory photographs of the effect of an ingrown-nail correcting method using a nail softener according to one embodiment of the present invention, particularly of a process of fixing a nail using resin, wherein FIG. 4(a) shows a nail before a treatment, and FIG. 4(b) shows the nail after the treatment.

FIG. 5 is a perspective view of an enclosure for external chemicals, according to a first embodiment of the present invention.

FIG. 6 is a sectional view taken along the line A-A in FIG. 5.

FIG. 7 is a sectional view of an enclosure for external chemicals, according to a second embodiment of the present invention.

FIG. 8 is a sectional view of an enclosure for external chemicals, according to a third embodiment of the present invention.

FIG. 9 is a top plan view of an enclosure for external chemicals, according to a fourth embodiment of the present invention.

FIG. 10 is a sectional view taken along the line B-B in FIG. 9.

FIG. 11 is an explanatory diagram of an ingrown-nail correcting method using an enclosure for external chemicals according to one embodiment of the present invention, particularly of a process of forming holes in a nail.

FIG. 12 is an explanatory diagram of an ingrown-nail correcting method using an enclosure for external chemicals according to one embodiment of the present invention, particularly of a process of attaching a wire to a nail.

FIG. 13 is a diagram showing the state after the wire is attached to the nail.

FIG. 14 is an explanatory diagram of an operation for attaching the enclosure according to the fourth embodiment to a nail.

FIG. 15 is an explanatory diagram of an operation for attaching the enclosure according to the fourth embodiment to the nail.

FIG. 16 is an explanatory diagram of an operation for fixing the nail using resin.

FIG. 17 is a diagram showing the state after the resin is removed from the nail.

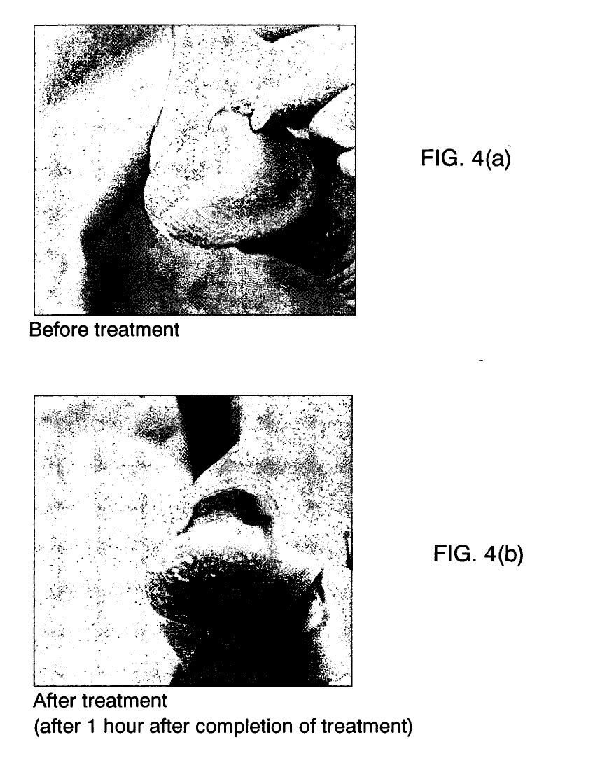

FIG. 4 is explanatory photographs of the effect of an ingrown-nail correcting method using a nail softener according to one embodiment of the present invention, wherein FIG. 4(a) shows a nail before a treatment, FIG. 4(b) showing the nail immediately after the treatment, and FIG. 4(c) showing the nail after one day from the treatment.

### DESCRIPTION OF THE PREFERRED EMBODIMENT

[Nail Softener]

A nail softener for use in deformed-nail corrective treatment or nail-structure modification (hereinafter referred to as “nail softener”) according to the present invention contains at least one reducing agent selected from the group consisting of cysteine, thioglycolic acid and thioglycolate.

The thioglycolate may include ammonium thioglycolate, sodium thioglycolate, potassium thioglycolate, monoethanolamine thioglycolate, diethanolamine thioglycolate, and triethanolamine thioglycolate. Preferably, the thioglycolate is ammonium thioglycolate.

The nail softener according to the present invention is preferably in the form of cream, paste or get which is prepared by adding an emulsifying substance to the above reducing agent. The emulsifying substance may include an emulsifying substance used in the first agent for permanent wave in the form of cream or the like, and any other suitable emulsifying substance having no adverse affect on a human body and the action of the reducing agent.

The nail softener may be appropriately added with an additive. Preferably, the additive is carotene, keratin, protein or calcium, which serves as nutrition of a nail.

In the nail softener according to the present invention, the concentration of the reducing agent is preferably set in the range of 2 to 50% by weight, depending on the type of reducing agent. If the concentration of the reducing agent is less than 2% by weight, it will be likely to cause difficulty in sufficiently softening a nail in a short period of time. If the concentration of the reducing agent is greater than 50% by weight, it will be likely to damage the nail adversely.

The nail softener according to the present invention may be added with a deodorizer for cleaning away odors peculiar to the reducing agent to be used, and various components serving as nutrition of a nail.

[Enclosure]

With reference to the drawings, an enclosure for external chemicals (hereinafter referred to as “enclosure” for brevity) according to an embodiment of the present invention will be described below. An external chemical to be enclosed in the enclosure according to the present invention may be any external chemical required for maintaining the contact with a diseased area, such as the aforementioned nail softener, a curative drug including an antifungal drug or antimycosis drug, or a mixture thereof. The external chemical is preferably in the form of gel, cream or paste.

### FIRST EMBODIMENT

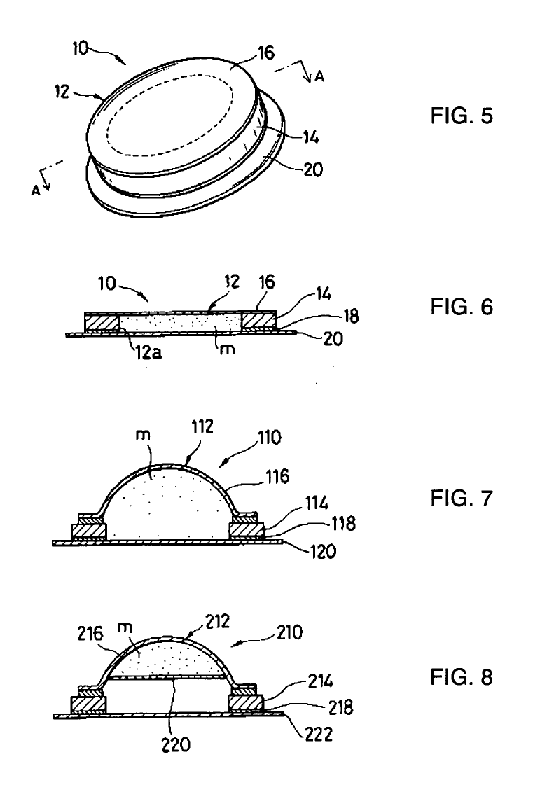

FIG. 5 is a perspective view of an enclosure according to a first embodiment of the present invention, and FIG. 6 is a sectional view taken along the line A-A in FIG. 5.

The enclosure 10 comprises an enclosure body 12 receiving an external chemical m therein and having an opening 12a for exposing the external chemical when used. The enclosure body 12 includes a ring-shaped pad 14 made of hard sponge or the like, and an upper film 16 attached with an adhesive or fusion-bonded on the top surface of the ring-shaped pad 14. As used in the specification, the term “ring-shaped” means any shape having an internal space.

An adhesive layer 18 is provided on the bottom surface of the ring-shaped pad 14. A sealing film 20 is releasably attached to the ring-shaped pad 14 through the adhesive layer 18 so as to allow the external chemical m to be sealingly enclosed in the enclosure body 12. This sealing film 20 also serves as a releasable film.

In this enclosure 10, the sealing film 20 can be removed to allow the external chemical m enclosed in the enclosure body 12 to be exposed to the outside thereof through the opening 12a. In this exposed state, the enclosure body 12 is attached to a diseased area by use of the adhesive layer 1 in such a manner that the external chemical is brought into contact with the diseased area to perform a curative treatment.

The amount of the external chemical to be enclosed in the enclosure body is set at a value allowing the external chemical to be applied onto the diseased area preferably at a thickness of at least 1 mm, more preferably about 2 mm or more, particularly about 3 to 7 mm.

### SECOND EMBODIMENT

FIG. 7 is a sectional view of an enclosure 110 according a second embodiment of the present invention.

The enclosure 110 comprises an enclosure body 112 receiving an external chemical m therein and having an opening 112a for exposing the external chemical when used. The enclosure body 112 includes a ring-shaped pad 114 made of hard sponge or the like, and a pocket 116 formed of a plastic film attached with an adhesive or fusion-bonded on the top surface of the ring-shaped pad 114. A plastic to be used in the plastic film is preferably transparent. This enclosure 110 can enclose the eternal chemical at a larger amount than that of the external chemical in the enclosure 10 according to the first embodiment.

An adhesive layer 118 is provided on the bottom surface of the ring-shaped pad 114. A sealing film 120 is releasably attached to the ring-shaped pad 114 through the adhesive layer 118 so as to allow the external chemical m to be sealingly enclosed in the enclosure body 112. This sealing film 120 also serves as a releasable film.

### THIRD EMBODIMENT

FIG. 8 is a sectional view of an enclosure 210 according a third embodiment of the present invention.

The enclosure 210 comprises an enclosure body 212 receiving an external chemical m therein and having an opening 212a for exposing the external chemical when used. The enclosure body 212 includes a ring-shaped pad 214 made of hard sponge or the like, and a pocket 216 formed of a plastic film attached with an adhesive or fusion-bonded on the top surface of the ring-shaped pad 214. As shown in FIG. 4, a sealing film 220 is attached to the inner surface of the pocket 216 through fusion bonding or the like. In the enclosure 210 according to the third embodiment, the external chemical m is sealingly enclosed in a space between the sealing film 220 and the pocket 216. This sealing film 220 is formed such that it is readily broken by pressing the pocket 16 inwardly.

An adhesive layer 218 is provided on the bottom surface of the ring-shaped pad 214. A releasable film 222 is releasably attached to the enclosure body 210 through the adhesive layer 218.

### FOURTH EMBODIMENT

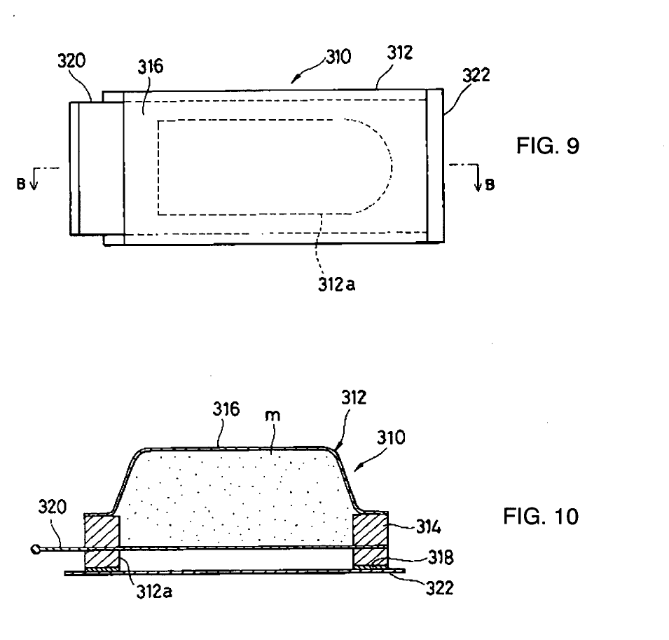

FIG. 9 is an enlarged top plan view of an enclosure 310 according a fourth embodiment of the present invention, and FIG. 10 is a sectional view taken along the line B-B in FIG. 9.

The enclosure 310 comprises an enclosure body 312 receiving an external chemical m therein and having an opening 312a for exposing the external chemical when used. The enclosure body 312 includes a ring-shaped pad 314 made of hard sponge or the like, and a pocket 316 formed of a plastic film attached with an adhesive or fusion-bonded on the top surface of the ring-shaped pad 314. As shown in FIG. 10, a sealing film 320 is interposed in the ring-shaped pad 314 in such a manner that it can be pulled out of the ring-shaped pad 314.

In the enclosure 310 according to the fourth embodiment, the external chemical m is sealingly enclosed in a space defined by the sealing film 320, the upper portion of the ring-shaped pad 314 and the pocket 316.

An adhesive layer 318 is provided on the bottom surface of the ring-shaped pad 314. A releasable film 322 is releasably attached to the enclosure body 310 through the adhesive layer 318.

In the above enclosures according to first to fourth embodiments of the present invention, when the aforementioned nail softener is used as the external chemical, the opening of the enclosure (ring-shaped pad) preferably has a shape precisely corresponding to that of a nail, particularly to that of a big toenail. Preferably, the opening of the enclosure (ring-shaped pad) has a shape and size capable of preventing the external chemical in the enclosure from being attached onto a skin around the periphery of a nail, if possible.

Further, the entire enclosure is preferably made of a material, such as plastic, having high water repellency.

[Method of Correcting Deformed Nail]

### FIRST EXAMPLE

With reference to the drawings, a deformed-nail correcting method using the nail softener according to one embodiment of the present invention will be described below.

The cases of ingrown nails include one case of a sufficiently grown nail, and another case of a deep nail. While the deformed-nail correcting method using the nail softener of the present invention can be applied to both the cases, the first example will be described in conjunction with one case where the method is applied to a sufficiently grown nail.

First Step (Preparation of Nail Softener)

An emulsifying agent was first added to ammonium thioglycolate serving as a reducing agent to prepare a 5% (weight %) ammonium thioglycolate mail softener in the form of cream or paste.

Second Step (Formation of Holes in Nail)

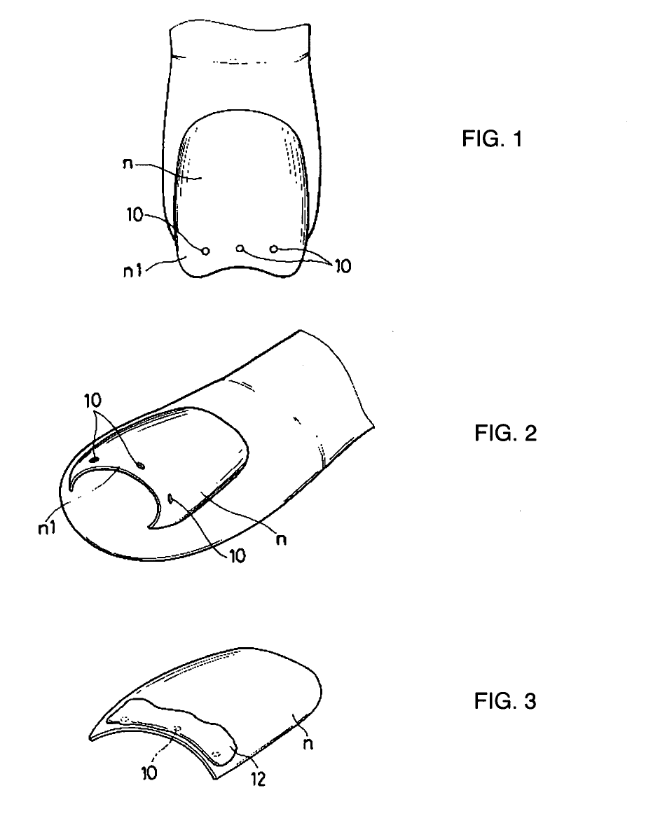

As shown in FIGS. 1 and 2, three small through-holes 10 were formed in the frontwardly extending region n1 of a nail n using a mini-rooter and a drill blade. The number of the small through-hole may be changed according to the state of the nail, and may be, for example, two.

Third Step (Application of Nail Softener to Nail)

The prepared nail softener was applied onto the entire surface of the nail at a thickness of about 5 mm, and left the nail untouched.

Fourth Step (Removal of Nail Softener from Nail)

After 25 minutes from the application of the nail softener to the nail, the state of the nail was checked. As a result, the nail was sufficiently softened. Thus, the nail softener was washed away and removed from the nail using a hot water.

Fifth Step (Operation of Repositioning Nail)

The softened ingrown nail was repositioned and corrected to its normal state (the state where the nail has a normal or relatively small curvature) by fingertips of an operator.

Sixth Step (Immobilization Using Resin)

In order to maintain the corrected position, a resin 12 was attached onto the upper surface of the nail to close the small through-holes 10 formed in the front region of the nail (see FIG. 3). This immobilization is performed for a period of time allowing the corrected position of the nail to be sufficiently maintained. While the period is dependent on the state of the nail, it was one hour in this example. An ultra-fast cure type autopolymerizing resin (GC Unifast II: trade name of GC Co.) was used as the resin.

Seventh Step (Final Step: Removal of Resin)

The resin was finally removed, and the correction of the nail was completed.

The result of the above corrective treatment is clearly indicated by photographs in FIG. 4. In FIG. 4, FIG. 4(a) shows the nail before the treatment, and FIG. 4(b) shows the nail after one hour from the completion of the treatment. While the nail before the treatment has a large deformation, it is repositioned and corrected by the action of the nail softener in a short period of time (1 hour) which is impossible to be achieved by the conventional techniques.

In the same process as that in the above example, corrective treatments were performed using another thioglycolate, cysteine and thioglycolic acid, respectively. While the corrective treatments were slightly different in the treatment period, substantially the same result as that in the above example could be obtained. The effect of the present invention can be verified based on the above results.

### SECOND EXAMPLE

With reference to the drawings, an ingrown-nail correcting method using the enclosure according to one embodiment of the present invention will be described below.

As mentioned above, the cases of ingrown nails include one case of a sufficiently grown nail, and another case of a deep nail. While the deformed-nail correcting method using the enclosure of the present invention can be applied to both the cases, the second example will be described in conjunction with one case where the method is applied to a deep nail.

First Step (Preparation of Nail Softener & Fabrication of Enclosure)

An emulsifying agent was first added to ammonium thioglycolate serving as a reducing agent to prepare a 5% (weight %) ammonium thioglycolate mail softener in the form of cream or paste. Then, an enclosure having the structure illustrated in FIGS. 9 and 10 and enclosing the prepared nail softener as an external chemical was fabricated

Second Step (Formation of Holes in Nail)

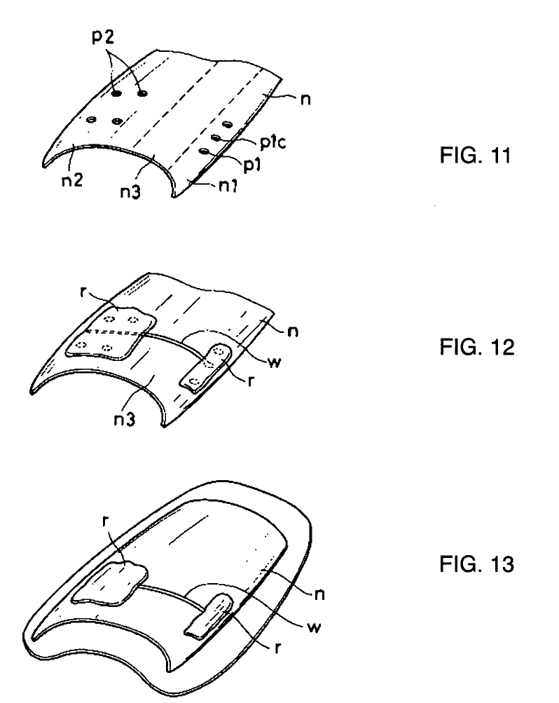

As shown in FIG. 11, using a mini-rooter and a drill blade, three small holes p1 were formed in one line along the longitudinal direction of a nail in one n1 of the lateral side regions of the nail n (the deep nail is caused on one of the lateral sides in many cases), and only the central one p1c of the holes was formed as a through-hole. The through-hole P1c was formed to fix one of the ends of a superelastic alloy wire w (see the aforementioned Patent Publication). Further, four small holes P2 serving as anchoring holes of resin r for fixing the other end of the wire were formed in the other lateral side region n2 of the nail.

Third Step (Setup of Superelastic Ally Wire: FIGS. 12 and 13)

One of the ends of the wire w is inserted into the central through-hole p1c. Then, the wire w was folded toward the other side region n2 or a flat region, and brought into close contact with the surface of the nail. A resin r was applied onto the curved region n1 to fix one end of the wire w. An ultra-fast cure type autopolymerizing resin (GC Unifast II: trade name of GC Co.) was used as the resin.

Then, the other end of the wire was fixed to the other lateral side region n2 or a flat region by use of a resin r. No rein was applied to the transitional (intermediate) region between the curved and flat regions. Then, the marginal portion of the wire was cut off.

Fourth Step (Setup of Enclosure Fabricated in Step 1)

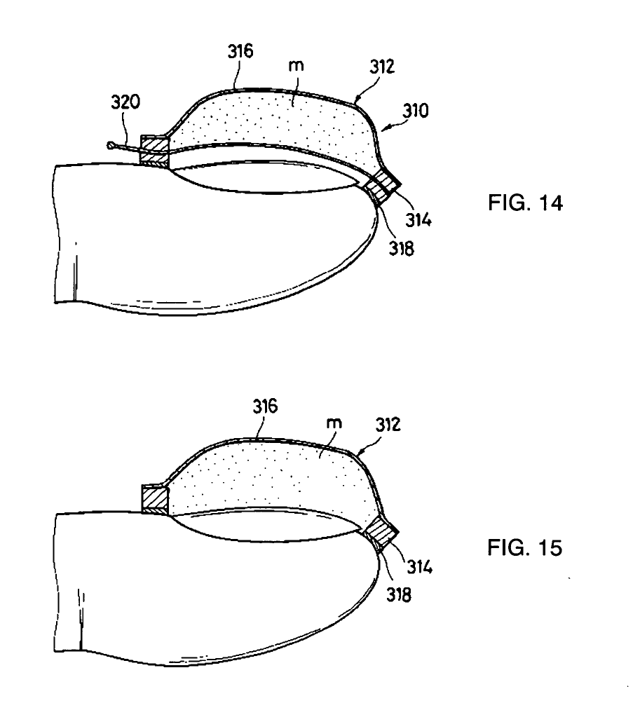

In order to cover the surface of the nail by the enclosure containing the nail softener for softening the nail so as to facilitate reposition of the nail, after removing the releasable film, the enclosure was attached to the nail by use of the adhesive layer (see FIG. 14). Subsequently, the sealing film was pulled out of the enclosure as shown in FIG. 15, and the pocket was pressed to allow the nail softener in the enclosure to be sufficiently brought into contact with the nail. This state was maintained for a given period of time. During this process, the nail was softened, and the ingrown nail was effectively repositioned by the action of the wire.

Fifth Step (Removal of Enclosure and Nail Softener from Nail)

After 2 hours from the application of the nail softener to the nail, the state of the nail was checked. As a result, the nail was sufficiently softened. Thus, the enclosure was detached from the nail, and the nail softener was washed away and removed from the nail using a hot water.

Sixth Step (Removal of Resin and Wire)

Then, the resin and the wire were carefully detached from the surface of the nail in a repositioned position using a small-size flat-head screwdriver.

Seventh Step (Immobilization Using Resin)

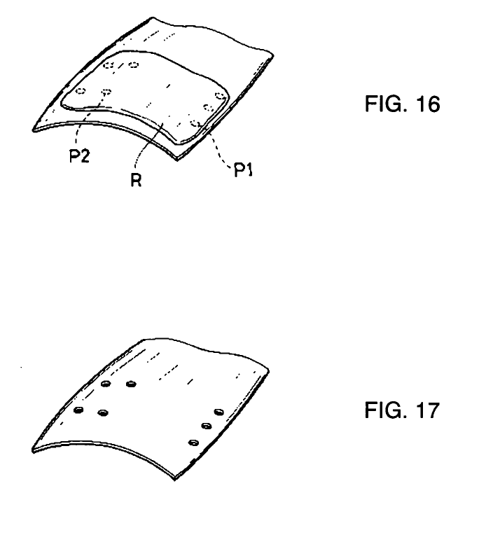

In order to maintain the corrected position, a resin R was fixedly attached over the wide range of the upper surface of the nail to close the small holes p1 and p2 previously formed in the nail (see FIG. 16). This immobilization is performed for a period of time allowing the corrected position of the nail to be sufficiently maintained. While the period is dependent on the state of the nail, it was two weeks in this example. The same ultra-fast cure type autopolymerizing resin (GC Unifast II: trade name of GC Co.) as that in the first example was used as the resin.

Right Step (Final Step: Removal of Resin)

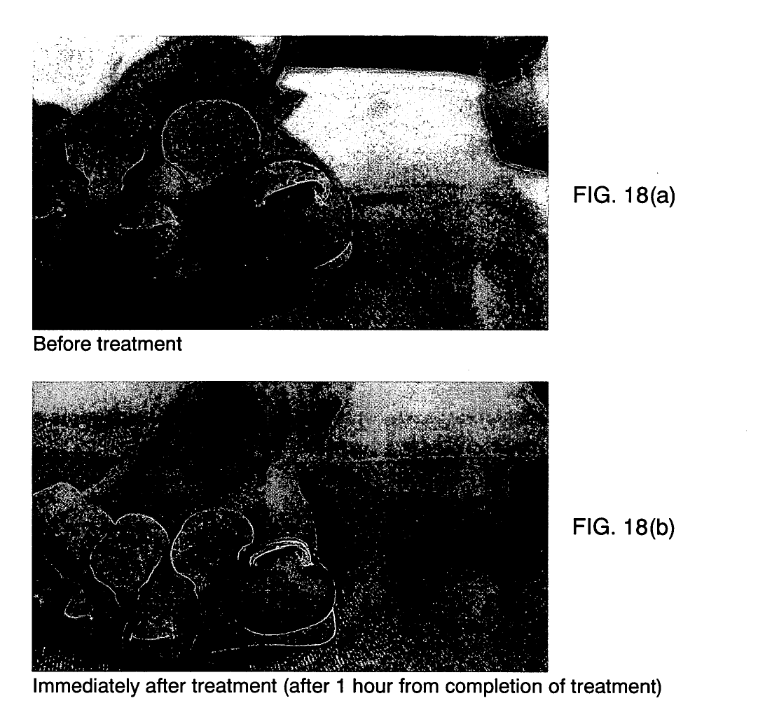

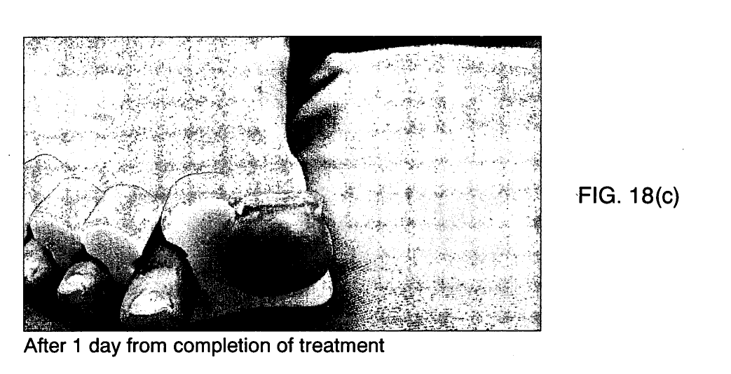

The resin was finally removed, and the correction of the nail was completed (FIG. 17).

The result of the above corrective treatment is clearly indicated by photographs in FIG. 18. In FIG. 18, FIG. 4(a) shows the nail before the treatment, FIG. 4(b) showing the nail immediately after the completion of the treatment, and FIG, 4(c) showing the nail after one day from the completion of the treatment. In the same process as that in the above example, corrective treatments were performed using another thioglycolate, cysteine and thioglycolic acid, respectively. While the corrective treatments were slightly different in the treatment period, substantially the same result as that in the above example could be obtained. The effect of the present invention can be verified based on the above results.

Advantageous embodiments and examples of the invention have been shown and described. It is obvious to those skilled in the art that various changes and modifications may be made therein without departing from the spirit and scope thereof as set forth in appended claims.

What is claimed is:

1. A nail softener for correcting a deformed nail or modifying the structure of a nail modification, containing at least one reducing agent selected from the group consisting of cysteine, thioglycolic acid and thioglycolate.

2. The nail softener as defined in claim 1, wherein said reducing agent is thioglycolate.

3. The nail softener as defined in claim 2, wherein said reducing agent is ammonium thioglycolate.

4. The nail softener as defined in claim 1, wherein said reducing agent is subjected to a deodorizing treatment.

5. An enclosure for an external chemical, comprising:
an enclosure body receiving said external chemical therein and having an opening for exposing said external chemical when used;
a sealing film for allowing said external chemical to be sealingly enclosed in said external chemical;
an adhesive layer provided at the peripheral portion of said opening of said enclosure body; and
a releasable film releasably attached to said enclosure body by use of said adhesive layer,
wherein after said releasable film is removed, said adhesive layer serves as an adhesive portion for allowing said enclosure body to be attached to a diseased area.

6. The enclosure as defined in claim 5, wherein said sealing film is attached to said enclosure body by use of said adhesive layer so that said releasable film is served by said sealing film.

7. The enclosure as defined in claim 5, wherein said sealing film is adapted to be broken or pulled out of said enclosure body so as to allow said external chemical enclosed in said enclosure body to be exposed to the outside thereof.

8. The enclosure as defined in claim 5, wherein said opening has a size equal to or greater than that of said diseased area.

9. The enclosure as defined in claim 5, wherein said enclosure body includes a ring-shaped pad, and an external-chemical enclosing pocket located on the top surface of said ring-shaped pad.

10. The enclosure as defined in claim 5, wherein said external chemical contains at least one reducing agent selected from the group consisting of cysteine, thioglycolic acid and thioglycolate.

11. The enclosure as defined in claim 10, wherein said reducing agent is thioglycolate.

12. The enclosure as defined in claim 11, wherein said reducing agent is ammonium thioglycolate.

13. The enclosure as defined in claim 10, wherein said reducing agent is subjected to a deodorizing treatment.

14. The enclosure as defined in claim 5, wherein said external chemical is a mixture of at least one reducing agent selected from the group consisting of cysteine, thioglycolic acid and thioglycolate, and a curative drug for said diseased area.

15. The enclosure as defined in claim 5, wherein said external chemical is a curative drug for said diseased area.

16. A method of correcting a deformed nail, comprising the steps of:
applying a nail softener containing at least one reducing agent selected from the group consisting of cysteine, thioglycolic acid and thioglycolate, onto a diseased area of a toenail;
leaving said diseased area untouched for a given period of time to soften said nail:
during or after said leaving step, applying a given force onto said nail to reposition said nail to its normal state; and
immobilizing said nail in said repositioned state for a given period of time to hold said repositioned state.
